(19) United States
(12) Patent Application Publication
(10) Pub. No.: US 2012/0219638 A1
(43) Pub. Date: Aug. 30, 2012

(54) Method and Compositions for Treating Plant Infections

(76) Inventors: Merle E. Olson, Calgary (CA); Michael W. Harding, Rosemary (CA)

(21) Appl. No.: 13/301,230

(22) Filed: Nov. 21, 2011

Related U.S. Application Data
(63) Continuation-in-part of application No. 11/913,158, filed on Feb. 12, 2010.
(60) Provisional application No. 60/815,723, filed on Jun. 22, 2006.

(51) Int. Cl. A01N 59/16; C01G 5/00; A01P 1/00; A01N 55/02; C07F 1/10

(52) U.S. Cl. 424/618; 514/495; 556/110; 423/395; 423/544

(57) ABSTRACT

The present invention includes antimicrobial silver-containing compositions that are effective in treating Erwinia species bacteria. These compositions are particularly effective for treating plants susceptible to Erwinia species infections.

The present invention is a continuing application of U.S. Ser. No. 11/913,158 filed 30 Oct. 2007; which is a national stage application of International Application No. PCT/CA2007/001149 filed 22 Jun. 2007; which claims the benefit of U.S. provisional application Ser. No. 60/815,723 filed 22 Jun. 2006. These applications are hereby incorporated by reference.

### I. FIELD OF INVENTION

This invention relates to compositions and methods for treating microorganisms and/or the diseases mediated by plant infections, and/or for treating, preventing, or reducing microbial contamination of plants, particularly trees, shrubs, bushes, including their flowers and fruiting bodies. The compositions and methods comprise at least one high valency silver-containing compound (e.g., Ag(II) and/or Ag(III)), in addition to Ag(I).

### II. BACKGROUND OF THE INVENTION

Environmental, medical, and industrial microbiologists have documented that microbial populations in their natural environments do not routinely grow as solitary or planktonic cells, but rather as biofilms; complex communities attached to surfaces (Costerton et al., 1995; Davey and O'Toole, 2003). These discoveries have shifted the conceptual framework for treating a wide variety of microbiological diseases and conditions, including but not limited to plant pathology (Marques et al., 2002; Dow et al., 2002; Ramey et al., 2004); and a wide variety of agricultural and farming applications; including but not limited to Pierce's Disease in grapes; potato ring rot and storage rots; browning root rot; fireblight; and seed infestations.

Plant diseases cause world-wide economic losses in all industries involving agricultural plant production including food commodity production, horticulture, floriculture, nutraceuticals, turf-grass, forages, nursery crops, forestry operations, fiber crop production, and alternative fuels. In addition, pathogens attack plant materials in post-harvest storages. Global economic losses due to plant diseases were estimated at 10%-15% reduction in potential production resulting in a cost of $76.1 billion between 1988 and 1990 (Orke et al., 1994; Pinstrup-Anderson, 2001). These infections in plants and produce are caused predominantly by microorganisms such as fungi, bacteria, nematodes, protists, and viruses.

Conventional commercial washing and sanitizing methods to remove microbial contaminants have been found to be marginally effective when biofilm is involved.

Bacterial and fungal pathogens can cause disease and loss to every sector of agriculture. For example, fire blight caused by the bacterium Erwinia amylovora is a devastating disease of susceptible fruit trees and ornamental trees/shrubs in the Rosaceae family worldwide resulting in millions of dollars (US) in losses. Management of the disease is mostly by treatments using the antibiotics streptomycin or oxytetracycline or by removing infected trees. In many parts of the US and in other countries, antibiotic-resistant isolates of E. amylovora have emerged in orchards making management of the disease even more difficult. Infections about which growers are most concerned occur in the spring during bloom. If infections during this stage can be eliminated or at least significantly reduced, yield and tree losses could be greatly reduced. Pears are the most susceptible, but apples, loquat, crabapples, quinces, hawthorn, cotoneaster, pyracantha, raspberry, and some other rosaceous plants are also vulnerable.

One particularly devastating bacterial pathogen is fire blight caused by the bacterium Erwinia amylovora. This bacterium is related to microorganisms which cause soft rot diseases such as E. carotovora and E. chrysanthemi, and the genus of Pantoea such as P. stewartii which causes Stewart's wilt in corn and hervicola. Fire blight infection is characterized by wilting and tissue necrosis. Fire blight itself affects many varieties of commercially important pome fruit trees—many varieties of apple and pear trees are particularly susceptible to fire blight. Other susceptible species include various varieties of stone fruit trees and some ornamental plants.

Affected areas of plants with fire blight appear scorched and blackened, symptoms which give fire blight its common name. Symptoms vary with the susceptibility of the plant and environmental conditions. Effects range from the destruction of specific plant structures to the death of the entire plant. For a more detailed discussion of fire blight, the reader is directed to an article by J. A. Eastgate, Molecular Plant Pathology (2000) 1 (6), 325-329, and references therein.

The microorganism which causes fire blight generally enters a susceptible plant through one of five primary routes for infestation. These include: formation of a canker; through a blossom; through new root or shoot tissue; or in response to trauma, damage caused by storms, or by human or animal activity. One common pathway to infection is through over-wintering of the organism in the bark of trees or in a canker on or in the bark. Cankers can be small and are easily overlooked during the winter pruning efforts. During the spring, the pathogen may ooze from the canker in a sticky, sap-like liquid which is readily dispersed by rain, wind, and pollinating insects such as bees. Once dispersed, the pathogen may infect blossoms of the same or neighboring plants. Infection of the blossoms is commonly referred to as blossom blight.

Blossom blight represents one particularly devastating form of the infection. Once the first infected stamen appears, pollinators, wind, and rain can rapidly carry the pathogen from one bloom to another. An entire orchard can be colonized within a several hours or a few days; when environmental conditions are suitable, the pathogen population can double within 20 to 30 minutes. Consequently, fire blight has been known to spread exponentially through stands of susceptible plants. When conditions favor the pathogen, it may sweep across an entire apple or pear tree orchard in a matter of only two to three days.

Once fire blight has infected a portion of a plant, growers must act aggressively to isolate the infected portion or in some cases the entire plant. Failure to take the appropriate remedial action immediately may result in loss of the entire orchard or surrounding nurseries. The most common approach to trying to control an outbreak of fire blight is to aggressively prune and in some cases completely destroy the infected plants. It is also common practice to destroy nearby non-infected plants in order to reduce the risk that the infection may spread into the entire orchard.

Additional approaches to fire blight control include treating plants with copper salts and antibiotics such as streptomycin and/or oxytetracycline. Since treatment after infestation is not always effective, nurseries often resort to prophylactically treating entire orchards with antibiotics to try and reduce the susceptibility of their crops to fire blight. As a result there have been reports of increased amounts of antibiotic residues in fruits, insects, and the soil around some orchards treated for fire blight. Furthermore, the widespread use of antibiotics has helped to select for variants of E. amylovora that are resistance to the antibiotics commonly used against E. amylovora. Despite these efforts, epidemics of fire blight appear to explode in orchards, many of which have no known history of infestation with fire blight. Clearly then, currently used methodologies for the control of fire blight are not particularly effective.

Once diseases are introduced, the only method of control is the application of registered chemical pesticides or pruning.

A complicating factor in plant pathology is the ability of pathogenic bacteria to form biofilms, which are often highly resistant to removal and disinfection (Costerton et al. 1999; Ceri et al. 2001; Olson et al. 2002). As a result, past and current experimental results may dramatically overestimate the efficacy of chemicals used as antimicrobial cleaners, pesticides, or disinfectants. It has been demonstrated that many bacterial pathogens can and do form biofilms either in vitro or on seed or plant surfaces (unpublished), resulting in current pesticide treatments being ineffective or marginally effective.

There is still a need for an effective antimicrobial and/or anti-biofilm agent with the following properties: inexpensive (or cost-effective), broad-spectrum efficacy, sustained release of anti-biofilm agent, ability to remove or degrade biofilms, and a low level of toxicity. This would be extremely beneficial to a very perishable commodity by lowering costs of disease management, increasing quality and economic value of plant material, increasing customer satisfaction, increasing consumer confidence, and promoting industry growth in the agricultural field as well as in the medical and industrial fields.

It is known in the art to employ methods and compositions comprising silver as an antimicrobial agent. The prior art, however, teaches use of silver as an antimicrobial agent against solitary or planktonic cells and not as an anti-biofilm agent against microorganisms growing as biofilms. It is known that covering a growing plant with silver nitrate provides an antimicrobial effect which helps protect the plant from disease. Also, the prior art teaches using monovalent silver as an antimicrobial agent but does not teach using silver of higher valency for treating plants or preventing plant diseases. Applicants have shown that high valency silver compositions are more effective as an anti-microbial and/or anti-biofilm treatment than silver nitrate (see for example, PCT/CA2010/002007, filed 14 Dec. 2010), incorporated herein by reference.

Having Ag(II) and Ag(III) makes the compounds of the present invention functionally or chemically distinct from the prior art because Ag(III) and Ag(II) are more reactive than Ag(I), resulting in higher efficacy and/or higher speed of kill. They may also help prevent development of bacterial resistance, because the more species the bacteria have to develop resistance to at once, the lower the probability that resistance will develop. Finally, the solid silver compounds of the present invention typically provide much better activity than monovalent silver in part due to the difference in solubility (the compounds of the present invention release low quantities of silver relatively slowly).

### III. SUMMARY OF THE INVENTION

There is a need for methods and compositions for treating, preventing, or reducing microbial contamination of plants, including but not limited to treating, preventing, or reducing microorganisms growing as biofilms on plants.

The compositions and methods of the present invention comprise high valency silver-containing compounds (e.g., containing Ag(II) and/or Ag(III)) as the anti-microbial agent.

We primarily teach silver (II) and silver (III)—high valency silver-containing complexes; versus high valency ions (silver²⁺ and silver³⁺). A chemical dictionary defines an ion as “an isolated electron or molecule that has acquired a net electric charge.” The same dictionary defines valence as “a positive number that characterizes the combining power of an element for other elements, as measured by the number of bonds to other atoms the given element forms upon chemical combination.” (Dictionary of Chemistry, McGraw-Hill, 1997).

The compositions and methods of the present invention have applicability in a wide variety of agricultural, industrial, and commercial environments (e.g., treating plant diseases and conditions; and disinfecting any surface, particularly disinfecting work or processing surfaces (e.g., tables) and seed or plant surfaces). The compositions of the present invention may also be used as an antimicrobial coating.

### IV. DETAILED DESCRIPTION OF THE INVENTION

The compositions and methods of the present invention comprise high valency silver-containing compounds (e.g., Ag(II) and/or Ag(III) in addition to Ag(I)) as the antimicrobial agent. These high valency silver-containing agents are the active agents in antimicrobial compositions. In preferred embodiments of the invention, the high valency silver compounds are one or more oxysilver nitrates, oxysilver bisulphates, other oxysilver compounds, or mixtures of these.

In some embodiments of the invention, the composition is a solid and contains oxysilver bisulphate, oxysilver nitrate, or a mix. Typically, this composition is a mixture of Ag (I), Ag (II), and Ag (III). For example, some compositions comprise one Ag (I), two Ag (II), and four Ag (III).

The compositions and methods of the present invention are also effective in treating and/or eradicating biofilm.

The compositions and methods of the present invention also include an active agent that inhibits the growth of microorganisms. As described in more detail below, the methods and compositions of the present invention may be used wherever biofilm or similar structures may be found, including but not limited to microorganisms growing and/or floating in liquid environments. In preferred embodiments of the invention, the compositions and methods may be used to treat, reduce, eradicate, or prevent growth of Erwinia species bacteria.

In some embodiments of the invention, the compositions and methods are used for treating a microbial contaminant using an antimicrobial agent comprising high valency silver. The compositions and methods may also include one or more other active agents, including but not limited to one or more additional antimicrobial agents, biological control agents (e.g., a bacterium that competes with the first bacterium), lubricants, preservatives, dispersants, and/or combinations thereof. Specific examples of some of these other active agents are disclosed below.

The present invention also comprises compositions and methods for treating a biofilm using an anti-biofilm agent comprising high valency silver-containing compounds (e.g., compounds containing Ag(II) and Ag(III)). The compositions and methods may also include one or more other active agents. The compositions and methods are antimicrobial, e.g., against biofilm, similar structures, or precursors formed by bacteria, fungi, viruses, algae, mollusks, parasites, yeast, and other microbes. In some embodiments of the invention, the antimicrobial effectiveness also applies to planktonic microorganisms. As described in more detail below, the methods and compositions of the present invention may be used wherever microbial infection or biofilm or similar structures may be found.

In some embodiments of the invention, the compositions and methods may be used to treat or prevent one or more biofilms. In some embodiments of the invention, the compositions and methods may be used to treat and/or prevent one or more plant diseases, conditions, infections, or contaminations. Typically these diseases and infections, etc., are caused by microbes associated with or residing in the biofilm.

The present invention also comprises compositions and methods to treat, prevent, or reduce one or more biofilms growing on a plant or portion thereof, using at least one form of high valency silver, such as, for example but not limited, to silver species having Ag (II) and Ag (III) valent states. In one embodiment, the method comprises treating, preventing, or reducing biofilm(s) on plant material by contacting the plant material with an anti-biofilm agent comprising at least one form of a high valency silver. In one embodiment, the composition may comprise an anti-biofilm agent comprising at least one form of a high valency silver. In the most preferred embodiments, the composition includes oxysilver nitrate and/or oxysilver bisulphate as the antimicrobial and/or anti-biofilm agent.

In some embodiments, the present invention comprises compositions and methods for preserving the health, life, or quality of plant material, including treating against bacteria, fungi, algae, biofilms, viruses, and parasites by contacting the plant material with a composition comprising one or more antimicrobial or anti-biofilm agents. These agents comprise one or more high valency silver species. In some embodiments of the invention, the compositions and methods may be used to preserve and/or disinfect plants, plant material, or parts thereof, most preferably blossoms. In some embodiments of the invention, the agent reduces or eliminates surface contamination.

In some embodiments of the invention, the high valency silver may be incorporated into or onto the packaging, shipping container, wrapper, or the like.

The present invention includes any method of contacting a plant or portion thereof with an antimicrobial agent or an anti-biofilm agent. Typical mechanisms of contacting include but are not limited to coating, spraying, immersing, wiping, and diffusing in liquid, gel, powder, or other delivery forms (e.g., injection, tablets, washes). In some embodiments of the invention, the compositions and methods may include applying the antimicrobial agent to any portion of a plant or plant material. Further, any structure or hard surface (e.g., tools or machinery surfaces associated with harvesting, transport, handling, packaging or processing) can be sanitized, disinfected, impregnated, or coated with the antimicrobial agent of the present invention.

Further, any storage or greenhouse facilities, or transport container can be impregnated with an antimicrobial agent of the present invention so that the antimicrobial agent prevents surface contamination and comes into contact with a plant or a portion thereof.

The compositions of the present invention may be used to treat a plant or portion thereof to prevent, eliminate, or reduce one or more undesirable and/or deleterious microorganisms. In these embodiments of the invention, the preservative compositions and methods may be an antimicrobial agent.

This invention demonstrates that stable, slow release high valency silver-containing compounds can be used as antimicrobials against bacterial and fungal pathogens, including biofilms, growing on plant surfaces or more broadly any surfaces associated with bacterial and fungal contaminants, e.g., wood, bark, leaves, concrete, metal, rubber, or plastic.

High valency silver, as used herein, refers to a compound or complex containing silver having valent states higher than one, such as, for example, Ag (II) and Ag (III) valent states. The preferred composition is an aqueous solution, aqueous slurry, or solid, more preferably one which gradually releases high valency silver-containing species when contacted by a solvent, diluents, suspending agent, or carrier. The compositions and methods of the invention may be comprised of silver having more than one valent state so that the composition containing the silver species may include multivalent substances. Finally, it is believed that the compositions of the present invention may be comprised of a silver-containing substance or a plurality of silver containing substances that react over time to form other silver containing substances which may exhibit differing antimicrobial properties.

Compositions of the present invention include any silver containing compound that produces a high valency silver species. These oxidized silver species include, for example, silver oxy-salts such as Ag₇O₈X, where X can include, but is not limited to NO₃⁻, ClO₄⁻, SO₄²⁻, F⁻, HSO₄⁻, Cl⁻, PO₄³⁻, CO₃²⁻, C₆H₅O₇³⁻, C₄H₄O₆²⁻, C₂O₄²⁻, etc. (Note: In the case of 2- or 3-charged anions, the number of silver in the oxysilver salt is 8 or 9, respectively, rather than 7), and their breakdown products, which may include silver(I) oxides (Ag₂O), higher silver oxides, i.e. Ag(II), and Ag(III) (AgO, Ag₂O₃, Ag₃O₄, or the like), and the silver(I) species including the anion present in the oxysilver compound (e.g. AgNO₃, AgClO₄, Ag₂SO₄, AgF, etc.). The preferred composition of the present invention comprises an aqueous suspension of an oxysilver compound, primarily or predominantly with active silver species Ag(II) and Ag(III).

These compositions exhibit antimicrobial activity and/or anti-biofilm activity against a variety of microbes, including both bacteria and fungi, and provide a sustained release of silver ions from silver compounds. The term “oxidized silver species” or oxysilver as used herein may involve but is not limited to compounds of silver where said silver is in +I, +II or +III valent states or any combinations thereof. The composition may also include elemental silver, preferably in small amounts, as a by-product of the oxidation production process.

Under certain conditions (e.g., exposure to heat and water), these compounds may also break down to form other high valency silver compounds or silver(I)-containing compounds which also have activity.

Applicants have also found that by manipulating production procedures in a conventional manner (e.g., changing time, temperature, pressure) the compounds of the present invention may also break down to form other high valency silver compounds or silver(I)-containing compounds that also have activity.

For example, the oxysilver compounds of the present invention may break down in the presence of water or heat to form AgO (which contains equal quantities of Ag(I) and Ag(III)) and a silver(I) species containing the appropriate anion (e.g., for oxysilver nitrate, silver(I) nitrate is formed; for oxysilver bisulphate, silver(I) sulphate is formed, etc.). These breakdown products also have antimicrobial activity.

In preferred embodiments of the invention, antimicrobial properties may be achieved by contacting an antimicrobially active high valency silver species within or at the surface of a substrate, such as a plant or portion thereof.

Another embodiment includes a spray or drench application about the locus of the plants including, for example, spraying the ground around the plant, particularly from the trunk or stem out to the drip line and/or injecting an aqueous solution of the control formulation into the ground around or under the plants or near the plant roots.

In one embodiment, an application of a formulation for the control or suppression of a plant pathogen can include dusting the exposed portions of a plant with a solid or powdered composition comprising the formulation. Still another embodiment includes applying the powdered composition to the ground around the plants or in the ground under the plants.

The spraying primarily of a liquid preparation of the formulation can be accomplished by a variety of methods including, but not limited to, blast sprayers, hose reel and hand guns, walking sprays, aerial sprays, and the like.

One embodiment provides formulations for the prophylactic treatment of plants prior to exposure to an infectious agent, or after confirmed or suspected exposure, but before the plant become symptomatic for an infection. Still another embodiment provides control of a pathogen by applying the formulation to plants which exhibit the symptoms and/or other evidence of infection of bacterial or fungal plant pathogens. Still another embodiment can control an infestation of plant pathogen by reducing the amount of damage done to the plant and by at least slowing the rate at which the infestation spreads to other parts of the host plant.

For suppression of a plant pathogen, a prophylactic treatment application can be made before the first signs of infestation or when environmental conditions appear to favor an outbreak.

In one embodiment, the formulation is applied to plants as required and in conformity with all governmental mandates and laws. In one embodiment, an aqueous spray formulation is applied prophylactically before the first appearance of flower or in early to full bloom. The prophylactic treatment can be repeated as desired or deemed expedient based upon the environmental conditions and/or the observance of bacterial infestation of neighboring plants, fields or orchards.

High valency silver may be produced by any process or reaction that produces high valency silver. The preferred processes are those that result in an aqueous suspension of the high valency silver. These processes are well known to those of ordinary skill in the art. See for example, J. A. MacMillan, Chem. Rev., 62, 65 (1962); S. S. Djokic, J. Electrochemical. Soc. 151, (6) C359 (2004); T. Nishimura and S. Hoshoda, Can. Metal. Quart. 47.1, 27 (2008); and G. I. N. Waterhouse et al., Polyhedron, 26, 3310 (2007).

In the preferred embodiments, the high valency silver-containing compounds or complexes may be produced by generating an aqueous solution of a monovalent silver salt or a silver complex such as silver nitrate or silver sulphate and oxidizing it. In preferred embodiments, the oxidizing agent is potassium persulfate (KPS) or ozone.

Alternative methods of producing high valency silver-containing species are well known to those skilled in the art.

The silver compounds may be used in any of the following formats: coatings, liquid, powder, capsule, tablet, and similar configurations. In a preferred embodiment of the present invention, active agents may be incorporated directly, or may be incorporated by sequentially adding components or precursors of the active agent to the plant material, and having the precursors of the active agent in or on the coating. Other forms also include films, sheets, fibers, sprays, and gels.

In preferred embodiments of the invention, the active agent in its solid form is predominantly Ag (II) and Ag (III), with small quantities of Ag(I) present. The inventors believe that high valency ions (e.g., Ag2+ and Ag3+) may be present in solution at some stages of plant treatment, primarily because one skilled in the art might expect compounds containing high valency silver to release ions into solution either by dissociation or by breakdown of the complexes containing the high valency silver. The latter is believed to be the case for oxysilver compounds.

The antimicrobial agents may be used for a variety of applications where there is a need for the presence of an antimicrobial agent. A preferred use is in the treatment and preservation of a plant, including but not limited to edible and fiber crops, fruit trees, produce, ornamental, nursery plants, tree seedlings, fiber plants, turf grass and forages, oilseeds, cereals, pulses, vegetables, medicinal plants, nutraceutical plants, and greenhouse crops.

The composition may also include additional antimicrobial agents, including but not limited to antifungal agents, antibacterial agents, anti-viral agents, antiparasitic agents, growth factors, angiogenic factors, anaesthetics, mucopolysaccharides, metals, disinfectants, antibiotics, cleaners, pH controllers (e.g., for providing an acidic growing environment) and other chemicals; and proteins or polypeptides (e.g., see U.S. Pat. No. 6,855,683). The active agents of the present invention may be used in combination with a cultivar or transgenic cultivar that is resistant to fire blight (see for example, U.S. Pat. No. 6,100,453).

Examples of antimicrobial agents that may be used in the present invention include, but are not limited to: 8-hydroxyquinoline sulfate, 8-hydroxyquinoline citrate, aluminum sulfate, quaternary ammonium, isoniazid, ethambutol, pyrazinamnide, streptomycin, clofazimine, rifabutin, fluoroquinolones, ofloxacin, sparfloxacin, rifampin, azithromycin, clarithromycin, dapsone, tetracycline, erythromycin, ciprofloxacin, doxycycline, ampicillin, amphotericin B, ketoconazole, fluconazole, pyrimethamine, sulfadiazine, clindamycin, lincomycin, pentamidine, atovaquone, paromomycin, diclazaril, acyclovir, trifluorouridine, foscamet, penicillin, gentamicin, ganciclovir, iatroconazole, miconazole, Zn-pyrithione, heavy metals (including, but not limited to, gold, platinum, silver, zinc, and copper), and their combined forms, including salts such as chloride, bromide, iodide, and periodate; complexes with carriers; and other forms.

For treating Erwinia species, the preferred additional antimicrobial agent is streptomycin, oxytetracycline or terramycin. Further, the first antimicrobial agent (the silver compound) and/or the second antimicrobial agent (e.g., the antibiotic) may be combined with or used in conjunction with a biological control agent. A biological control agent is a microorganism that competes with or itself kills the undesirable microorganism, and includes, but is not limited to, any beneficial bacterium that is not killed by the silver and antibiotics. An exemplary biological control agent that discloses treating Erwinia species may be found in U.S. Pat. No. 8,025,875, and U.S. Pat. No. 7,906,131, both incorporated herein by reference.

The composition may also include known plant or seed treatments and fungicidal products such as Vitaflo 280, Apron-Maxx RTA, and thiram. The composition may also include one or more seed coatings, enhancers, emulsifiers, thickening agents, solvents, anti-foaming agents, preservatives, fragrances, coloring agents, emollients, fillers, and the like. These and other ingredients, including inactive ingredients, in the composition are conventional and well known to those skilled in the art.

Treating biofilm, as used herein, refers to contacting a biofilm or similar structure with an anti-biofilm agent wherever biofilm may be found, is expected to be found, or is postulated to be found. One skilled in the art will readily recognize that the areas and industries for which the present invention is applicable is a vast number of processes, products, and places. A particularly preferred use is in the treatment and preservation of plant materials in both the agricultural and horticultural sectors.

Another aspect is a method for controlling or suppressing plant pathogens which includes the steps of providing an aqueous formulation and applying the aqueous preparation to plants. The amount of the preparation applied to a given field or stand of plants can be expressed as the number of gallons of a standard preparation of the formulation applied per acre of planted area. In one embodiment, the aqueous preparation is applied in the amount of between 15 gallons to about 350 gallons per acre of area including the plants of interest. In another embodiment, a formulation not intended for treatment of certain plants that produce edible fruits or other edible structures may include, between about 2.5×10⁷ cfu to about 5.0×10⁷ cfu of at least one strain of Tricoderma selected form the group consisting of T. harzianum and T. konigii and between about 1.0×10⁸ to about 2.0×10⁸ cfu Baccillus licheniformis per one hundred gallons of an aqueous preparation.

In this aspect of the invention, the compositions and methods are suitable for treating one or more microbial infections, including but not limited to, diseases or conditions caused by Pseudomonas spp., Xanthomonas spp., Curtobacterium spp., Sclerotinia spp., Pythium spp., Fusarium spp., Botrytis cinerea , Helminthosporium solani, Streptomyces spp., Phytophthora spp., Rhizoctonia solani, Erwinia pp., and Clavibacter spp., to name just a few.

Exemplary disease or conditions include, but are not limited to bacterial blight, brown spot, common blight, vascular wilt, white mold, root rots, head blight, fire blight, silver scurf, dry rot, common scab, ring rot, soft rot, damping off, seedling blight, seed rot, and bacterial canker.

The compositions and methods of the present invention are also effective or beneficial as a protective coating and/or as an ingredient in a protective coating.

The formulations according to various embodiments may include at least one sticking agent. A sticking agent is a compound that has as at least one of its characteristics the ability to adhere to a surface structure of a plant or to at least one other component in a given formulation. Suitable sticking agents include, but are not limited to yucca plant extracts, Kaolin clay; fine wet-able powders, and the like.

The formulations according to various embodiments may include at least one agent or compound that at least helps to protect components of a given formulation from the damaging effects of ultraviolet (UV) radiation, or from rapid desiccation. These compounds include, but are not limited to fine clays, Kaolin clay, aluminum oxide, zinc oxide, aluminum silicate, and the like.

The formulations according to various embodiments may include at least one wetting agent. A wetting agent promotes the dispersal of the formulation in an aqueous environment. Wetting agents may also promote a more even, more efficient, spreading of various components in the formulation onto above-ground plant structures including, but not limited to, leaves, stems, petioles, bark, blossoms, fruits, and the like. Suitable wetting agents include, but are not limited to yucca plant extract.

In one embodiment, the method for treating a plant includes a spray or drench application of an aqueous preparation of a formulation, for the control or suppression of a plant pathogen, to the exposed surfaces of a plant, i.e., any part of the plant extending above ground. This includes the undersides, tops, or side surfaces of leaves, stems, trunk bark, buds, blossoms, flowers, fruits, and the like, or parts thereof.

### Definitions

As used herein, the term “plant” refers to any living organism belonging to the kingdom Plantae (i.e., any genus/species in the Plant Kingdom). This includes familiar organisms such as, but not limited to, trees, herbs, bushes, grasses, vines, ferns, mosses, and green algae. The term refers to both monocotyledonous plants, also called monocots, and dicotyledonous plants, also called dicots. Examples of particular plants include, but are not limited, to apple trees, citrus fruits (e.g. grapefruit, lemon, lime, orange, tangerine, citrus hybrids, pummelo, and other citrus fruit crops), stone fruits (e.g., coffees, jujubes, mangos, olives, coconuts, oil palms, pistachios, almonds, apricots, cherries, damsons, nectarines, peaches and plums), and the like. For a more complete list of representative crop plants see, for example, Glossary of Crop Science Terms: Ill, Nomenclature, Common and Scientific Names, Crop Science Society of America, July 1992, which is herein incorporated in its entirety.

As used herein, the term “plant part” refers to any part of a plant including, but not limited to, the shoot, root, stem, seeds, stolons, rhizomes, tubers, cut flowers, stipules, leaves, petals, flowers, blossoms, ovules, bracts, branches, petioles, internodes, bark, pubescence, tillers, rhizomes, fronds, blades, pollen, stamen, and the like. The two main parts of plants grown in some sort of media, such as soil, are often referred to as the “above-ground” part, also often referred to as the “shoots”, and the “below-ground” part, also often referred to as the “roots”. For a more comprehensive list of plant parts see, for example, James W. Perry and David Morton (1998) Photo Atlas for Botany, Wadsworth Publishing Company, which is herein incorporated in its entirety.

One skilled in the art will recognize that a biofilm may be composed of a single species, may be multi-species, homogenous, heterogeneous, and/or may also include other organisms associated with or protected by the biofilm. Biofilm as used herein also refers to one or more stages of biofilm development or formation.

During biofilm formation, microbes aggregate with each other or may adhere to a surface, encasing themselves in a self-produced matrix of extracellular polymers. This occurs in a tightly regulated response to environmental cues and results in physiological and genetic diversification of the cells in the biofilm. This cellular diversity is linked to an increase in antimicrobial resistance and tolerance of the microbial population. Because of this, biofilms are thought to be responsible for many chronic or device-related infections that are recalcitrant to personalized antibiotic therapy based on MIC testing.

As used herein, anti-biofilm agent refers to any element, chemical, biochemical, or the like that is effective against a biofilm. Typical anti-biofilm agents are those that have antimicrobial, anti-bacterial, anti-fungal or anti-algal properties, including when these species grow in biofilms. Metal and metal compounds, preferably high valency silver, have been shown generally to have antibacterial and ethylene inhibiting properties, and are preferred anti-biofilm agents in accordance with the present invention. In some embodiments of the invention, the anti-biofilm agent is a broad spectrum agent, e.g., having effectiveness or activity against more than one microbial species.

“Incorporating” as used herein refers to any process or composition involving at least one high valency silver-containing compound that results in high valency silver being biologically and/or medically available as antimicrobial agents.

“Sustained release” or “sustainable basis” are used to define release of atoms, molecules, ions, or clusters of a metal that continues over time measured in hours or days, and thus distinguishes release of such metal species from the bulk metal, which release such species at a rate and concentration which is too low to be effective, and from highly soluble salts of noble metals such as silver nitrate, which releases silver ions virtually instantly, but not continuously.

Planktonic, as used herein, refers to microorganisms growing as floating, single cells, which is part of their life cycle.

Surface contamination, as used herein, refers to microorganisms growing on or relocated to a surface. The microorganisms associated with surface contamination may be actively growing or dormant, but represent a viable inoculum that can reinitiate infection, disease, or other undesirable conditions.

Agriculture includes all sectors, commodities, and surfaces associated with plant and food production including but not limited to horticulture, field production, greenhouse production, nursery crops, turf and forages, fiber crops, alternative fuels, and forestry for all phases of production, transport, processing and packaging of plant-derived commodities used for food, fiber, landscaping, or recreation. Additionally, agriculture includes all aspects of production, transport, processing, and packaging of animal-derived commodities used for food or otherwise. This definition includes any natural or man-made surfaces associated with production, transport, handling, processing, and packaging of both plant- and animal-derived commodities.

Fire blight as used herein refers to an aggressive, devastating disease that infects several varieties of trees, including fruit trees such as apples and pears trees, as well as many members of the family Rosaceae. These include the following genera and species varieties including Amelanchier (serviceberry), Exchorda (pearlbush), Potentilla (cinquefoil), Aroina (chokeberry), Fragaria (strawberry), Prinsepia, Aruncus (goatsbeard), Ceum (avnes), Prunus (apricot, cherry, plum), Chaenomeles (flowering quince), Heteromeles (toyon), Pyracantha (firethorn), Cotoneaster (cottoneaster), Holodiscus (creambush), Pyrus (pear), Cowania (cliff rose), Kageneckia, Raphiolepes (Indian hawthorn), Crataegomespilus, Kerria (Japanese rose), Rhodotypos (jetbead), Crataegus (hawthorn), Malus (apple, crabapple), Rosa (rose), Cydonia (quince), Mespilus (medlar), Rubus (brambles), Dichotomanthes, Osteomeles, Sorbaria (false spirea), Docynia, Peraphyllum, Sorbus (mountain ash), Dryas (mountain avens), Photinia (photinia), Spiraea (spiraea), Eriobotrya (loquat), Physocarpus (ninebark) and Stranvaesia. While only affecting members of the rose family, the host range includes over 130 species and nearly 40 genera (Sinclair et al., Disease of Trees and Shrubs, Cornell University Press, 1987). Fire Blight Disease (FBD) first appeared in the north eastern parts of North America approximately 200 years ago. It has since spread to New Zealand in 1916, England in 1957, Egypt in 1962, and various regions of Europe (Bereswill et al., App. Env. Micro. 58 (1992), pp. 3522-3526; van des Zwet and Bell, HortScience 30(6) (1995), pp. 1287-1291).

The present invention will be further described in detail with reference to the following working examples.

### EXAMPLES

### Example 1

### Preparation of High Valency Silver Compound

High valency silver was prepared using known techniques, as follows: Oxysilver compounds (combination of oxysilver nitrate and oxysilver bisulphate) were prepared through the reaction of aqueous solutions of silver nitrate (AgNO₃) and potassium persulfate (K₂S₂O₈) to yield a black precipitate of oxysilver bisulphate and oxysilver nitrate. The precipitate is recovered by filtration and the powder is dried.

### Description of Starting Materials

| | |
|---|---|
| Silver Nitrate (AgNO₃) | Technical Grade |
| Potassium Persulfate (K₂S₂O₈) | Technical Grade |
| Water | Distilled |

A. To a clean 4 L Erlenmeyer flask, equipped with over-head stirrer, charge with de-ionized water (1.7 L).
B. Start the agitation and manually charge in small portions 75.6 g potassium persulfate (KPS).
C. Agitate the mixture until KPS is dissolved. Dissolution was verified and the pH was checked.
D. In a clean 4 L beaker (glass) prepare a mixture of de-ionized water (0.375 L) and silver nitrate (45 g). Agitate until dissolved. Check the pH of the solution.
E. To a second 4 L Erlenmeyer flask, charge 0.5 L deionized water and equip with an efficient overhead stirrer (the overhead stirrer in Step A can be transferred).
F. Connect the flask containing the potassium persulfate mixture (from Step A) and the 4 L beaker containing the silver nitrate solution (Step D) to the flask prepared in Step E using transfer lines and metering pumps. Agitate the water in the flask (from Step E) at high speed (e.g. 554 rpm).
G. Maintaining ambient temperature, transfer the silver nitrate solution and the KPS solution simultaneously to the water in the flask (from Step E). The KPS addition is started slightly ahead of silver nitrate addition, with the KPS being added at 50 mL/min and the silver nitrate being added at 10 mL/min. A black precipitate will begin to form immediately.
H. Maintain good agitation during the addition process, which should take roughly 1 h. Check the pH of the supernatant solution. The mixture should be strongly acidic.
I. Continue to agitate the reaction mixture for an additional 1 hour. Let the solids settle and decant or siphon off the bulk of the supernatant liquid into a flask (the one used in Step A is fine) and hold for later disposal.
J. Filter the aqueous slurry onto a suitably prepared filter nutsche (glass sintered is best) and pull dry the filter cake. Use a spatula to smooth out the filter cake to avoid cracking and channeling. Check the pH of the filtrate. Add the filtrate to the flask containing the supernatant (from Step I). Hold for disposal. Note: If necessary, material which may be held up in the reaction flask can be transferred to the filter using a small amount of water.
K. Slurry wash the filter cake with about 40-50 mL of deionized water and pull dry. Measure the pH of the filtrate. Repeat this operation if the filtrate is still acidic (pH<4). Note: Try not to go above pH 4, as the product becomes less stable at neutral pH. Add the wash water to a waste container (could be the container used above) and hold for disposal.
L. Pull dry the filter cake by keeping the system under vacuum for at least 3 minutes. Stir the filter cake frequently using a spatula and spread out the filter cake. This will break up larger aggregates and assists removal of water. Protect the filter cake from collecting dust or other foreign objects.
M. Discharge the filter cake to a suitable dryer (tray dryer or other container) and determine the weight of the wet material. Dry under a stream of air for 12 hours or in a desiccator over a drying agent such as active molecular sieves. Half-way through the drying period, check the material for lumps and break them up if needed. After 12 hours, remove a sample and analyze for water content following the SOP for water determination via Karl Fischer titration. Extend the drying period in approximately 6 hour increments as needed until the water content of the material is below 1%. If additional drying periods are required, record the results of each water analyses.
N. Transfer the dry product to a suitable tared, labeled container and determine the gross and net weight. Remove a sample for quality control. Place the product container in a glass container containing a desiccant pouch in the cold room.
O. Waste Disposal: The waste water (from decanting and filtration operations) is collected and combined as described above. The total volume of waste collected is recorded. A 50 mL sample of the waste is analyzed to determine the proper disposal path. The pH of the solution is checked. The sample is checked for active oxygen using KI paper. If the sample is still active, the waste is neutralized with base to ˜pH 5 and then any un-reacted persulphate is destroyed using a bisulfite solution. The active oxygen content is checked again, and additional bisulfite solution added as needed. Once the mixture contains no more active oxygen, it is disposed of to aqueous waste.

### EXAMPLE 2

### High Valency Silver Anti-Microbial Activity against Erwinia carotovora subsp. carotovora (Ecc), the Soft Rot of Vegetables Pathogen, in Comparison to Nanocrystalline Silver Powder

TABLE 1
Ecc biofilm susceptibility to high valency silver (Oxy) and
nanocrystalline powders Ag₃0 and Ag 100 (Nanotechnologies, Inc.)
at 24 h contact time. Cell counts are expressed in log₁₀, silver
compound concentration is in parts per million.

| | Ag₃0 | Ag100 | Oxy |
|---|---|---|---|
| 500 ppm | 0 | 0 | 0 |
| | 0 | 0 | 0 |
| | 0 | 0 | 0 |
| | 0.00 | 0.00 | 0.00 |
| 200 pm | 0 | 0 | 0 |
| | 0 | 0 | 0 |
| | 0 | 0 | 2.11 |
| | 0.00 | 0.00 | 0.70 |
| 100 ppm | 0 | 0 | 0 |
| | 0 | 0 | 0 |
| | 0 | 0 | 1.95 |
| | 0.00 | 0.00 | 0.65 |
| 50 ppm | 0 | 1.30 | 1.60 |
| | 1.60 | 0.00 | 1.00 |
| | 1.00 | 1.48 | 2.00 |
| | 0.87 | 0.93 | 1.53 |
| 0 ppm | 3.85 | 3.70 | 3.48 |
| | 3.78 | 3.60 | 3.90 |
| | 3.60 | 3.60 | 3.90 |
| | 3.74 | 3.63 | 3.76 |

TABLE 2
Log reduction of Ecc biofilms treated with high valency silver (Oxy) and
nanocrystalline powder Ag₃0 and Ag 100 at 24 h contact time.

| | Ag₃0 | Ag100 | Oxy |
|---|---|---|---|
| 500 ppm | 3.74 | 3.63 | 3.76 |
| 200 ppm | 3.74 | 3.63 | 3.06 |
| 100 ppm | 3.74 | 3.63 | 3.11 |
| 50 ppm | 2.87 | 2.7 | 2.23 |

### Conclusion

High valency silver was as efficacious as nanocrystalline silver as an antimicrobial against plant pathogenic Erwinia spp.

### EXAMPLE 3

Experimental design: Three cultivars, 8 treatments and four blocks. In each block there is one tree per cultivar per treatment (four trees per cultivar per treatment). Treatments are randomized within each cultivar in each block.

### Treatments:

1. Water control

2. Streptomycin-control

3. Oxysilver compound 0.005% (w/v); for 12 L=0.6 g

4. Oxysilver compound 0.05%; for 12 L=6 g

5. Oxysilver compound 0.1%; for 12 L=12 g

6. Oxysilver compound 0.5%; for 12 L=60 g

7. Oxysilver compound 1%; for 12 L=120 g

8. Biological control

When the forecast predicts favorable conditions, treatments were applied within 24-48 hours. Note: Oxysilver compound was a mixture of oxysilver bisulphate and oxysilver nitrate, with oxysilver bisulphate as the dominant compound.

Parameters evaluated: Disease incidence, severity, foliar, flower and fruit phytotoxicity data and total yield

### Methods:

Three apple varieties (Gala, Fuji, and Golden) were treated with one spray of oxysilver at full-bloom. There were a total of eight treatments: 1. Untreated control, 2. Streptomycin standard, 3. Oxytetracycline standard, 4. Oxysilver compound 0.005% (w/v), 5. Oxysilver compound 0.05%, 6. Oxysilver compound 0.1%, 7. Oxysilver compound 0.5%, and 8. Oxysilver compound 1.0%. The trees were evaluated once a week for disease severity and phytotoxicity. Apples were harvested. Each treatment and variety combination was repeated four times. Disease incidence through blossom infections was monitored. Infections four weeks or so after bloom are not due to blossom infections and are not typically treated by growers.

Disease incidence was recorded as the number of trees showing infections per treatment. Disease severity was rated as follows: 0—No disease, 1—One—two infected shoots, 2—Two-four infected shoots, 3—Four or more infected shoots or one systemic canker, 4—Two or more systemic cankers. The ratings for phytotoxicity of fruit were: 0—No phytotoxicity, 1—Occasional russetting, 2—Occasional deformation or increased russetting, 3—Severe russetting, 4—Severe deformation. The ratings for phytotoxicity of foliage were: 0—No phytotoxicity, 1—<10% of leaf area necrotic, 2—10-25% of leaf area necrotic, 3—25-50% of leaf area necrotic, 4—50-100% of leaf area necrotic. After harvest, apples from untreated trees and trees treated with 0.005% and 1% oxysilver compound were sent to an independent lab for testing of silver accumulation in the fruit. For comparison, oxysilver compound was sprayed directly on some apples.

Statistical analysis was done using SAS 9.3 Proc glimmix and Proc GLM.

### Results:

Treatments 4, 6, 7 and 8 had significantly lower incidence rates than the untreated control but were not significantly different from treatments 2, 3 and 6 (Table 1). Disease seventy was significantly higher in the untreated control in which systemic cankers occurred compared to all oxysilver treatments and the streptomycin standard (p=0.05).

Leaf phytotoxicity one week after treatment was significantly higher for Treatments 7 and 8 (0.5% oxysilver compound and 1.0% oxysilver compound, respectively). Leaves with phytotoxicity were replaced. New leaves did not show any signs of phytotoxicity. Phytotoxicity of fruit for Treatments 7 and 8 was severe and significantly higher compared to all other treatments. For statistical analysis of yield, only the yield data of ‘Gala’ and ‘Golden’ trees were used. The cultivar ‘Fuji’ is alternate year bearing and most trees had very little or no fruit. There were no significant differences in yield among the treatments (Table 1).

Apple samples were sent after harvest to an independent laboratory for testing for the presence of silver in the fruit. The highest concentration found was 0.06 ppm in a Fuji apple treated with 1.0% oxysilver compound at full bloom. Detection levels for apples from Treatment 4 with 0.005% oxysilver compound ranged from no silver detected to 0.03 ppm (see attached report).

Wash water from blossoms a week after treatment was plated and recovered E. amylovora from untreated ‘Gala’ blossoms (multiple E. amylovora colonies) and ‘Gala’ blossoms from Treatment 6 (one Erwinia amylovora colony). Other bacteria were isolated as well from all treatments except Treatments 7 and 8. There were no bacteria recovered from blossoms from Treatments 7 and 8 from all three varieties.

TABLE 1
Disease incidence, yield and phytotoxicity results for apples treated with
oxysilver compound at full bloom

| | Disease | | | |
|---|---|---|---|---|
| | incidence | Ave. | Phytotoxicity | Phytotoxicity |
| | (# of | yield/tree | (leaf) | (fruit) |
| Treatment | trees) | kg) | (average) | (average) |
| 1. Untreated control | 2 a* | 8.24 a | 0 c | 0 c |
| 2. Streptomycin | 1 ab | 6.85 a | 0 c | 0 c |
| standard | | | | |
| 3. Oxytetracycline | 1 ab | 7.64 a | 0 c | 0 c |
| standard | | | | |
| 4. Oxysilver | 0 b | 6.65 a | 0 c | 0 c |
| compound 0.005% | | | | |
| 5. Oxysilver | 1 ab | 7.90 a | 0 c | 0 c |
| compound 0.05% | | | | |
| 6. Oxysilver | 0 b | 7.27 a | 0 c | 0 c |
| compound 0.1% | | | | |
| 7. Oxysilver | 0 b | 7.16 a | 1.75 b | 2.8 b |
| compound 0.5% | | | | |
| 8. Oxysilver | 0 b | 8.74 a | 3.08 a | 1.3 a |
| compound 1.0% | | | | |

*Treatments with the same letter are not significantly different from each other.

### Conclusion:

Even though this was a light fire blight year, there were significant differences in disease incidence and disease severity between the oxysilver compound treatments and the untreated control. All oxysilver compound treatments performed as well or better than the streptomycin and oxytetracycline standards after being applied once during full bloom. Oxysilver compound treatments containing 0.005%-0.1% active ingredient show no phytotoxicity, whereas the treatments with 0.5% and 1% oxysilver compound showed severe foliar and fruit phytotoxicity. The phytotoxicity levels for the treatments with high concentrations of oxysilver compound are unacceptable for growers. Oxysilver compound has been very effective in this trial at very low concentration at which no phytotoxicity occurred and it is anticipated that continued trials will show its great potential for fire blight management.

We claim:

1. A method for treating plants of the family Rosaceae comprising contacting the plant or a portion thereof with a composition that comprises at least one antimicrobial agent, said antimicrobial agent comprising at least one compound containing high valency silver (e.g. Ag(II) and/or Ag(III)), thereby treating the plant.

2. The method claim 1 wherein the compound is an oxysilver compound.

3. The method claim 3 wherein the oxysilver compound is oxysilver nitrate or oxysilver bisulphate.

4. The method of claim 1 wherein treating the plant comprises treating the plant against one or more biofilms.

5. The method of claim 4 wherein treating the plant against one or more biofilms comprises preventing, eradicating, or reducing the biofilm.

6. The method of claim 4 wherein treating the plant comprises treating the plant material against one or more Erwinia species.

7. An antimicrobial composition comprising high valency silver-containing ions, complexes, or compounds, said ions, complexes, or compounds being effective against one or more Erwinia species.

8. A method for treating a plant susceptible to an Erwinia species infection, comprising contacting the plant or a portion thereof with a composition that comprises at least one anti-microbial agent, said anti-microbial agent comprising at least one compound containing high valency silver, thereby treating the plant.

9. The method of claim 8 wherein the compound is an oxysilver compound.

10. The method of claim 8 wherein the Erwinia species is Erwinia amylovora.

11. The method of claim 8 further comprising contacting the plant with at least one second anti-microbial agent, said agent selected from the group consisting of streptomycin, oxytetracycline, terramycin, and combinations or mixtures thereof.

12. The method of claim 2 wherein said oxysilver compound contains an anion selected from the group consisting of sulfates, nitrates, chlorides, chlorates, fluorides, phosphates, carbonates, citrates, tartrates, iodates, bisulphates, and oxalates; and mixtures or complexes thereof.

13. The method of claim 1 wherein the composition further comprises one or more additional active agents, which may include breakdown products of the oxysilver compounds.

14. The method of claim 13 wherein the additional active agents are selected from the group consisting of a biocontrol agent, streptomycin, oxytetracycline, terramycin, and combinations or mixtures thereof.

15. A method for inducing resistance to fire blight disease in a susceptible plant comprising administering to the plant at least one compound containing a high valency silver.

16. The method of claim 15 wherein the high valency silver contains Ag(II) and/or Ag(III).

17. The method of claim 15 wherein the high valency silver is an oxysilver compound.

18. The method of claim 15 wherein a susceptible plant is selected from the group consisting of genera and species varieties including Amelanchier (serviceberry), Exchorda (pearlbush), Potentilla (cinquefoil), Aroina (chokeberry), Fragaria (strawberry), Prinsepia, Aruncus (goatsbeard), Ceum (avnes), Prunus (apricot, cherry, plum), Chaenomeles (flowering quince), Heteromeles (toyon), Pyracantha (firethorn), Cotoneaster (cottoneaster), Holodiscus (creambush), Pyrus (pear), Cowania (cliff rose), Kageneckia, Raphiolepes (Indian hawthorn), Crataegomespilus, Kerria (Japanese rose), Rhodotypos (jetbead), Crataegus (hawthorn), Malus (apple, crabapple), Rosa (rose) Cydonia (quince), Mespilus (medlar), Rubus (brambles), Dichotomanthes, Osteomeles, Sorbaria (false spirea), Docynia, Peraphyllum, Sorbus (mountain ash), Dryas (mountain avens), Photinia (photinia), Spiraea (spiraea), Eriobotrya (loquat), Physocarpus (ninebark), and Stranvaesia.
